(19) United States
(12) Patent Application Publication
(10) Pub. No.: US 2019/0154648 A1
(43) Pub. Date: May 23, 2019

(54) METHODS OF ATTENUATING DRUG EXCIPIENT CROSS REACTIVITY

(71) Applicant: Chesapeake Therapeutics, LLC, Pikesville, MD (US)

(72) Inventors: Robb Lawrence, Owings Mills, MD (US); Robert E Bakin, Burlingame, CA (US)

(21) Appl. No.: 16/314,766

(22) PCT Filed: Jul. 19, 2017
(86) PCT No.: PCT/US17/42947

§ 371 Date: Jan. 2, 2019

Related U.S. Application Data
(60) Provisional application No. 62/602,132, filed on Apr. 13, 2017.
(60) Provisional application No. 62/364,438, filed on Jul. 20, 2016.

(51) Int. Cl. G01N 33/15; A61K 9/00

(52) U.S. Cl. CPC G01N 33/15; A61K 9/0048; A61K 9/0043; A61K 9/0019; A61K 9/0031; A61K 9/0034; A61K 9/0046

(57) ABSTRACT

The present application relates generally to novel pharmaceutical drug formulations comprising modified chemical compositions having reduced capacity for excipient mediated cross reactivity. Methods for their use and methods for identifying excipient related incompatibilities and/or adverse events are also disclosed.

### CROSS-REFERENCE TO RELATED APPLICATIONS

This application claims the benefit of U.S. Provisional Appl. No. 62/364,438, filed Jul. 20, 2016, and U.S. Provisional Appl. No. 62/602,132, filed Apr. 13, 2017. The content of the aforementioned applications is relied upon and is incorporated by reference in their entirety.

### FIELD OF THE INVENTION

The field of the invention relates to pharmaceuticals, particularly pharmaceutical formulations and methods of making the same.

### BACKGROUND OF THE INVENTION

Drug formulation efforts traditionally focus on identifying combinations of physiochemically compatible active pharmaceutical ingredients and drug excipients. Drug label safety warnings frequently highlight known or suspected interactions involving active pharmaceutical ingredients (e.g., cross reactivity with other active ingredients) as well as potentially harmful side effects of individual, stand-alone formulation excipients (e.g., iodine).

Drug-drug API interactions continue to impact patient safety, morbidity and mortality. Cross reactivity among most APIs are largely predictable (i.e., “on-label” side effects) and can be avoided. Far less predictable are “off-label” side effects having no known mechanistic basis. Similar to on-label side effects, off-label side effects are more common in genetically predisposed patient populations (see FIG. 3). It stands to reason that individuals receiving two or more medications (e.g., polypharmacy) are at an increased risk of not only predictable on-label side effects, but also unpredictable off-label adverse events. Individuals particularly susceptible to off-label adverse events also include pediatric populations, elderly, those with autoimmune disorders, those with gastrointestinal disorders, or those with other health problems.

Pharmaceutical excipients are generally known in the art to be chemical substances, other than the pharmacologically active drug or prodrug, included in the manufacturing process or are contained in a finished pharmaceutical product dosage form. Excipients account for most of the weight or volume of a medicinal product. Physical and chemical interactions between active pharmaceutical ingredients and excipients can affect the chemical nature, the stability and bioavailability of drug products, and consequently, their therapeutic efficacy and safety. Safety profiles of excipients are largely established on an individual excipient basis and acceptable levels of exposure are assessed based largely on an average healthy person. While excipients are often essential components of finished drug products, they are also potential toxicants as ether single agents or in combination with other excipients and/or active pharmaceutical ingredients. Representative non-limiting examples of individual excipient-induced toxicities include: renal failure and death from diethylene glycol, osmotic diarrhea caused by ingested mannitol, hypersensitivity reactions from lanolin, and cardiotoxicity induced by propylene glycol.

Chemically compatible excipients safely stabilize the active ingredient against environmental degradation, facilitate administration and can modulate release of the active pharmaceutical ingredient. Excipients are found in a variety of drug formulations, including, but not limited to: oral, intravenous, nasal, subcutaneous, buccal, topical, enteral, parenteral, epidural, intramuscular, intraocular, and other formulations generally known to those skilled in the art. Regulatory authorities classify excipients generally safe or “inert” when administered individually, to otherwise healthy patients and below certain temporal concentration thresholds.

Excipient concentrations above accepted safety thresholds are no longer inert chemicals from a regulatory perspective. Despite being generally classified as inert chemicals under all conditions, excipients (and/or their byproducts, breakdown products and manufacturing contaminants) can adversely affect the safety and/or efficacy of the API and/or final drug product and thus the patient. Indeed, the FDA is alerting drug and dietary supplement manufacturers, compounding pharmacies, and distributors of povidone analogs (e.g., povidone/copovidone/crospovidone), that the agency recently detected excessive levels of peroxide in one lot of crospovidone (cross linked polyvinyl N-pyrrolidone) manufactured overseas. Excipients from one drug formulation that are incompatible with the API and/or excipients in another drug formulation can give rise to iatrogenic effects in healthy individuals as well as those with preexisting disease FIG. 4. An emerging concern relates to newly discovered/synthesized excipients designed for modified-release formulations and/or the requirements of the modern high-productivity compressing/tableting machines that lack sufficient safety testing.

This background information is provided for informational purposes only. No admission is necessarily intended, nor should it be construed, that any of the preceding information constitutes prior art against the present invention.

There remains a need for improved, excipient-reduced or excipient-free drug formulations that minimize the likelihood of excipients contributing to adverse drug events. In particular, oral liquid formulations allow for a reduction in excipient levels normally associated with equivalent solid dosage formulations.

### SUMMARY

It is to be understood that both the foregoing general description of the embodiments and the following detailed description are exemplary, and thus do not restrict the scope of the embodiments.

In one aspect, the invention provides an excipient reduced pharmaceutical drug formulations obtained by: (i) comparing excipient profiles from two or more co-administered solid drug formulations, (ii) identifying the excipient or excipients common to said two or more co-administered solid drug formulations, (iii) reformulating one or more of said co-administered solid drug formulations without the excipient or excipients common to said two or more co-administered drug formulations.

In another aspect, the invention provides a method of identifying an excipient or excipients contributing to adverse events in an individual comprising comparing adverse events associated with co-administration of two or more drug pharmaceutical formulations having at least one common excipient to the adverse events associated with co-administration of two or more drug pharmaceutical formulations lacking said common excipient.

In another aspect, the invention provides a method of identifying an excipient or excipients contributing to adverse events in an individual comprising comparing adverse events associated with repeated daily administration of a drug pharmaceutical formulation containing an excipient or excipients to the adverse events associated with repeated daily administration of a drug pharmaceutical formulation having the same active ingredient but lacking said excipient or excipients.

In another aspect, the invention provides a method of identifying an excipient or excipients contributing to adverse events in a non-human animal comprising comparing adverse events associated with co-administration of two or more drug pharmaceutical formulations having at least one common excipient to the adverse events associated with co-administration of two or more drug pharmaceutical formulations lacking said common excipient.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Exemplary known active ingredient/excipient incompatibilities.

FIG. 2. Excipient incompatibilities with API's of different therapeutic classes.

FIG. 3. Genetic incidence rates of individual excipient toxicities.

FIG. 4. Known drug-excipient incompatibilities.

FIG. 5. Exemplary common drug pairs containing shared excipients in representative patient populations.

FIG. 6. Known excipient warnings in hypersensitive patient populations.

FIG. 7. Examples of OTC excipients capable of excipient stacking.

FIG. 8. Worldwide variability of excipient containing drug formulations.

### DETAILED DESCRIPTION

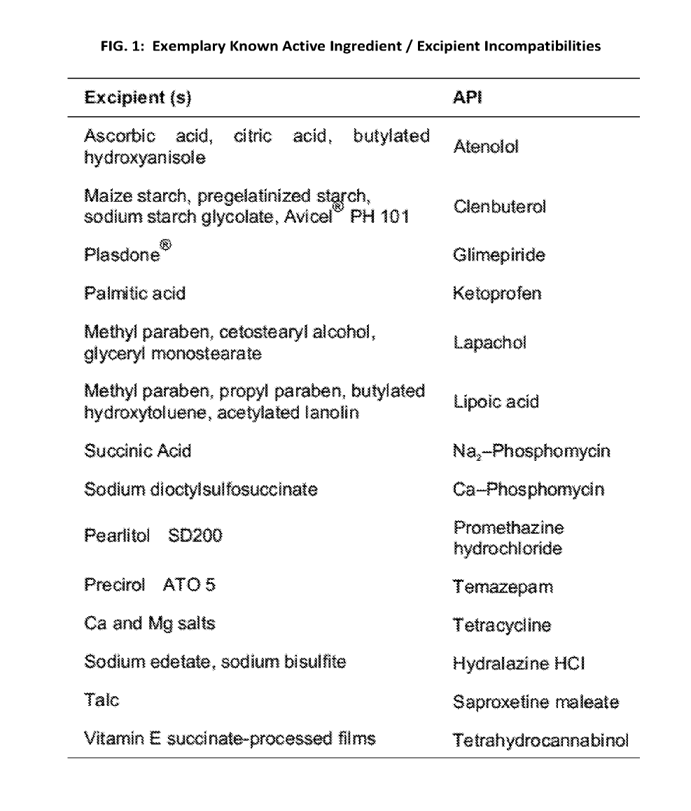

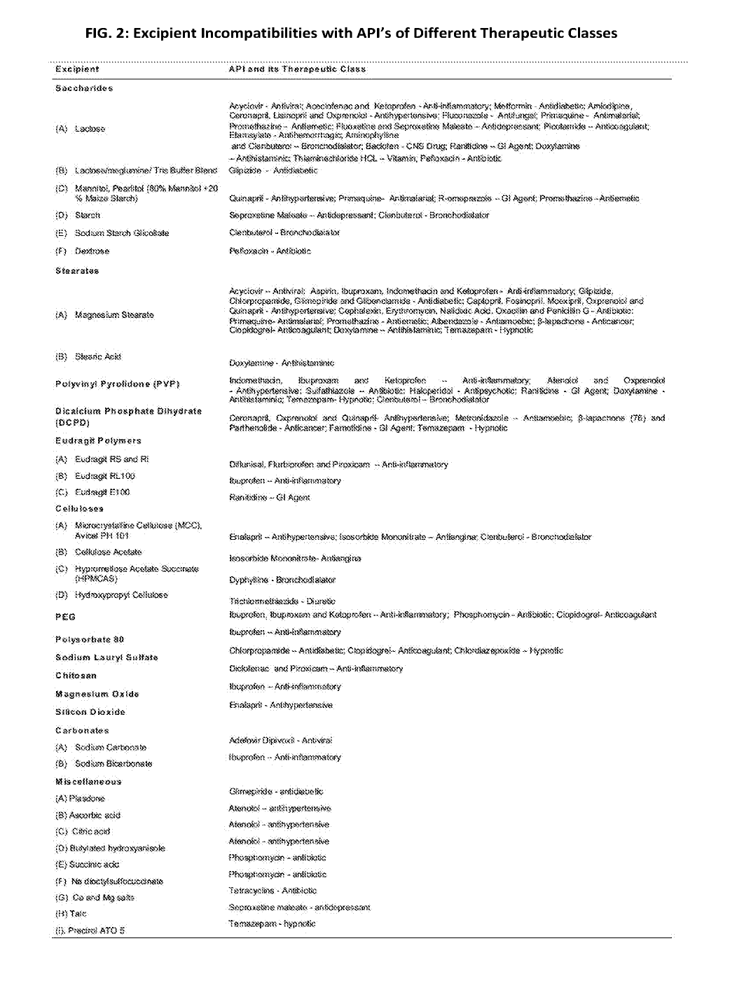

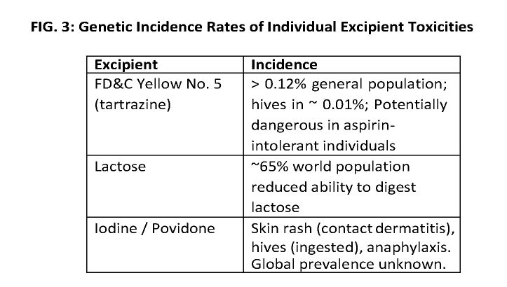

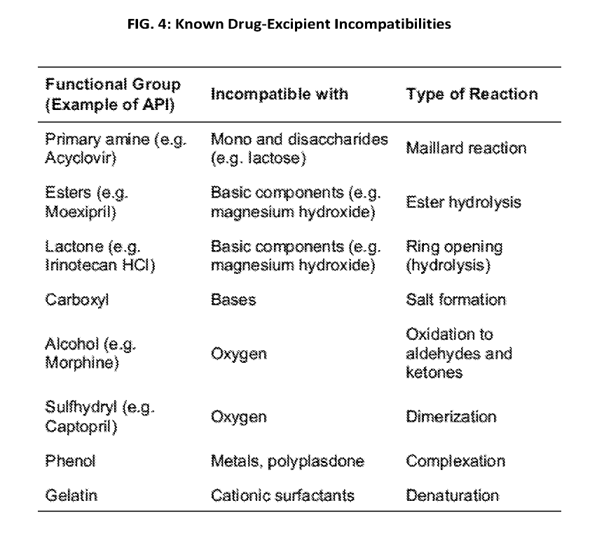

The present application relates generally to a previously unrecognized source of drug safety concern—combinatorial, excipient mediated adverse events. Adverse events not attributed to known active ingredient incompatibilities potentially have their origins in excipient incompatibilities. Toxic excipient mediated adverse interactions can occur via additive and/or synergistic mechanisms and can occur upon co-administration of two or more excipient containing drugs having presumably compatible active ingredients, but incompatible excipients. We generally refer to this effect as “excipient stacking”. By way of non-limiting examples, excipients from a first drug may be incompatible with excipients and/or active pharmaceutical ingredients (API) from a second drug FIGS. 1 and 2. By way of another example, excipient stacking may also occur when an excipient or excipients from one or more drug formulations are incompatible with the combination of two or more APIs from another drug formulation or formulations. Under this type of polypharmacy, the combination of two or more APIs contributes either a direct or indirect role for the emergence of excipient stacking, possibly by sensitizing the individual to the co-administered excipient or excipients. Another non-limiting example of excipient stacking would include the combination of two or more subtoxic doses of the same excipient(s) (e.g., from repeated use of the same drug or repeated use of a different drug with same excipient(s)) in such a manner as to create a toxic dose of the excipient. Other logical permutations of this concept are within the scope of this disclosure. To address this novel and previously unrecognized drug safety issue, the present application generally discloses methods of novel drug formulations substantially free of potentially cross-reactive excipients, methods of use for said drug formulations, and methods for identifying excipient related incompatibilities associated with polypharmacy.

In one representative and non-limiting embodiment, the present application discloses methods of identifying drug formulation components involved in excipient stacking. In another representative and non-limiting embodiment, the present application discloses methods of preparing reformulations of equivalent API solid dosage forms that lack excipients associated with excipient stacking and/or associated adverse events. In another representative embodiment, the present application identifies methods for identifying and avoiding undesirable excipient-excipient and/or excipient-drug iatrogenic events. In another representative embodiment, the present application discloses methods for treating patients who desire, or are otherwise in need of, excipient-modified or excipient-reduced drug formulations.

A general goal of this invention is increasing the awareness of the problem of excipient stacking and avoiding unintended adverse events due entirely, or in part, to excipient stacking. This can be accomplished by: (i) proactively developing novel, excipient-reduced or absent drug formulations with less likelihood of excipient stacking, (ii) developing methods of identifying problematic excipients during polypharmacy and (iii) developing methods of treatments to those patients who are susceptible to excipient stacking.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

The term excipient generally applies to non active-ingredient substances routinely used in formulating drug, food and/or cosmetic products as understood by a person of ordinary skill in the art. The term excipient as used herein also applies more broadly to process or product-related impurities (e.g., excipient degradation products, excipient leachates, excipient residual solvents, co-processed excipients) or extraneous contaminants. The term excipient also includes compositions or compounds that protect the activity or integrity of an active agent when the active agent is exposed to certain conditions (e.g., drying, freezing). In some embodiments, a protective agent protects an active agent during a freezing process (i.e., it is a “cryoprotectant”). Examples of protective agents include but are not limited to non-fat milk solids, trehalose, glycerol, betaine, sucrose, glucose, lactose, dextran, polyethylene glycol, sorbitol, mannitol, poly vinyl propylene, potassium glutamate, monosodium glutamate, Tween 20 detergent, Tween 80 detergent, and an amino acid hydrochloride.

The term excipient stacking generally refers to instances where an excipient or excipients from one or more drug formulations when in combination with another excipient, excipients and/or an active ingredient or ingredients from another drug formulation or formulations elicits an drug event not generally attributable to an active ingredient or ingredients. The drug event may be either adverse or beneficial to the individual.

The term iatrogenic generally refers to an illness or adverse event caused by medical examination or treatment that may or may not be directed or undertaken by a physician. For example, iatrogenic shall include an illness or adverse event caused by a person who is not a physician or healthcare professional who self-administers a drug (e.g. OTC drugs).

The term off label side effect refers to any side effect not already listed on the commercially marketed drug package insert.

The phrase therapeutically effective amount refers to an amount of an active pharmaceutical ingredient or drug, which is capable of performing the intended result.

The term polypharmacy refers generally to the simultaneous use of two or more drugs to treat one or more ailments or conditions. As defined herein, polypharmacy is not limited to prescription drugs and can include non-limiting examples such as over the counter (OTC) drugs or medications such as: cough drops/suppressants, herbals, vitamins, supplements, pain and/or fever reducers, cosmetics, skin care products, hair care products, toothpaste, oral care products, and other products commonly found in drug stores and pharmacies.

Co-administration of two or more excipient containing drug products (e.g., polypharmacy) containing the same (or different) excipient(s) and/or active pharmaceutical ingredient may result in iatrogenic effects not attributable to the active pharmaceutical ingredient or ingredients. Such unexpected effects may be desirable (e.g., better gut tolerability) or undesirable (e.g., anaphylaxis). Additional sources contributing to excipient stacking may arise from improper use of excipient containing products—such as parenteral instead of oral administration (e.g., of a pharmaceutical containing egg albumin). In light of this previously unrecognized source of drug formulation adverse events, excipients should be carefully evaluated when pharmaceuticals are re-formulated, especially in the case of modified-release formulations or for use in vulnerable patient populations (e.g., pediatric, psychiatric, elderly). Use of injectable ‘cocktails’ should be allowed only after careful verification of the chemical and physical compatibility of the active ingredients and their respective excipient containing solutions. Rare instances of excipient stacking may also occur via interactions between drug excipient and package/delivery device/capsule means.

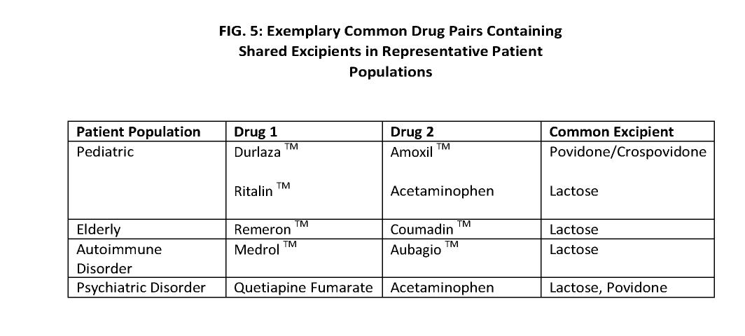

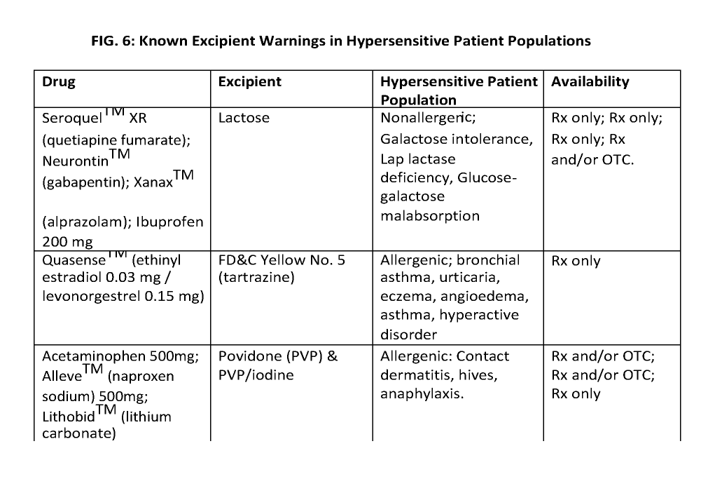

We recommend that drug developers undertake routine testing to ensure that excipient profiles of commonly co-administered drugs are compatible (e.g., will not demonstrate excipient stacking), particularly in vulnerable patient populations FIGS. 5 and 6.

Suggestively, while the antipsychotic quetiapine fumarate has low single-dose toxicity, repeat-dose toxicity studies in nonhuman primates demonstrate both anticipated API side effects of the central nervous system but also mechanistically unexplainable side effects including: pigmentation of the thyroid, transient increases in heart rate, posterior triangular cataracts, and reduced lymphocyte count. One cannot rule out that such observed iatrogenic effects are due, at least in part, to excipient stacking.

Examples of presumed API adverse interactions that may be alternatively attributed to excipients, at least in part, can be found at www.ScienceTranslationalMedicine.org 14 Mar. 2012 Vol. 4 Issue 125 and is hereby incorporated by reference. Building on this theme, a recent report looking for interactions between pairs of excipient containing drug formulations yielded far more side effects than could be attributed to active ingredients.

Meta analysis of the FDA adverse experience database (FAERS) database may identify previously unrecognized examples of excipient stacking in a small subset of the human population. Indeed, a range of published analyses of FAERS data demonstrate significantly more adverse drug experiences occurring than would be expected based on known individual active ingredient side effects, drug-drug interactions and/or hypersensitivities to single drug excipients.

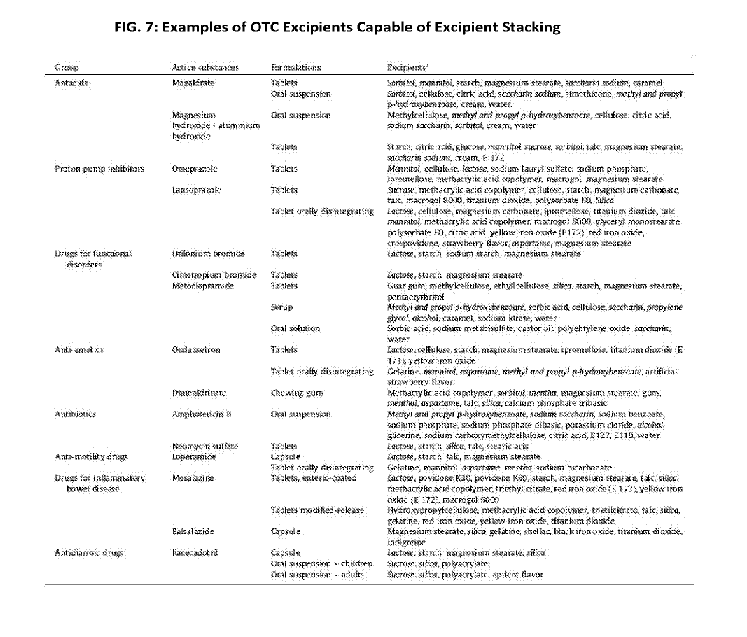

One of the earliest known examples of excipient toxicity occurred in 1937 in which the excipient diethylene glycol found in Elixir Sulfanilamide was responsible for the death of more than one hundred people. FDA is becoming increasingly aware that certain excipients can cause serious toxicities not only in prescription drugs, but over-the-counter (OTC) drugs. It should be noted that OTC drugs are particularly amenable to excipient stacking due to a lack of direct guidance from healthcare providers and/or repeated, long-term self-dosing (e.g., cough drops/suppressants, antihistamines, decongestants, etc.) FIG. 7.

Clinical trials occasionally perform excipient-controlled studies because of a possibility that drug excipients may be a confounding variable in drug performance and/or implicated in adverse outcomes. For example, one study compared the tolerability of omalizumab to omalizumab excipients in patients with allergic asthma. What have not been performed are control studies characterizing combinations of drug excipients from commonly co-administered drug formulations. Polypharmacy is particularly problematic for psychiatric conditions where multiple medications are often prescribed.

Food products would also be expected to contribute to excipient stacking. Excipients found in drug formulations are also commonly found in food products (e.g., lactose, tartrazine/FD&C Yellow No. 5.) The FDA Center for Food Safety and Applied Nutrition (CFSAN) maintains a database known as the Priority-based Assessment of Food Additives (PAFA). PAFA contains administrative, chemical and toxicological information on over 2,000 substances directly added to food, including substances regulated by the U.S. Food and Drug Administration (FDA) as direct, “secondary” direct, color additives, Generally Recognized As Safe (GRAS) and prior-sanctioned substances. The more than 3000 total substances together comprise an inventory often referred to as “Everything Added to Food in the United States” (EAFUS). The EAFUS list of substances contains ingredients added directly to food that FDA has either approved as food additives or listed or affirmed as GRAS. Nevertheless, it contains only a partial list of all food ingredients that may in fact be lawfully added to food, because under federal law some ingredients may be added to food under a GRAS determination made independently from the FDA. The list contains many, but not all, of the substances subject to independent GRAS determinations.

Similar to bona fide drug excipients, it should be noted that food excipients are evaluated individually. No combinatorial studies (e.g., food-drug or food-cosmetic) are routinely performed. In one embodiment, excipient stacking results from food-drug, food-cosmetic or food-drug-cosmetic interactions. By way of non-limiting examples, dietary supplements, nutritional supplements, nutraceuticals, probiotics, vitamins, cosmetics and other non-drug products containing excipients would also be expected to contribute to excipient stacking.

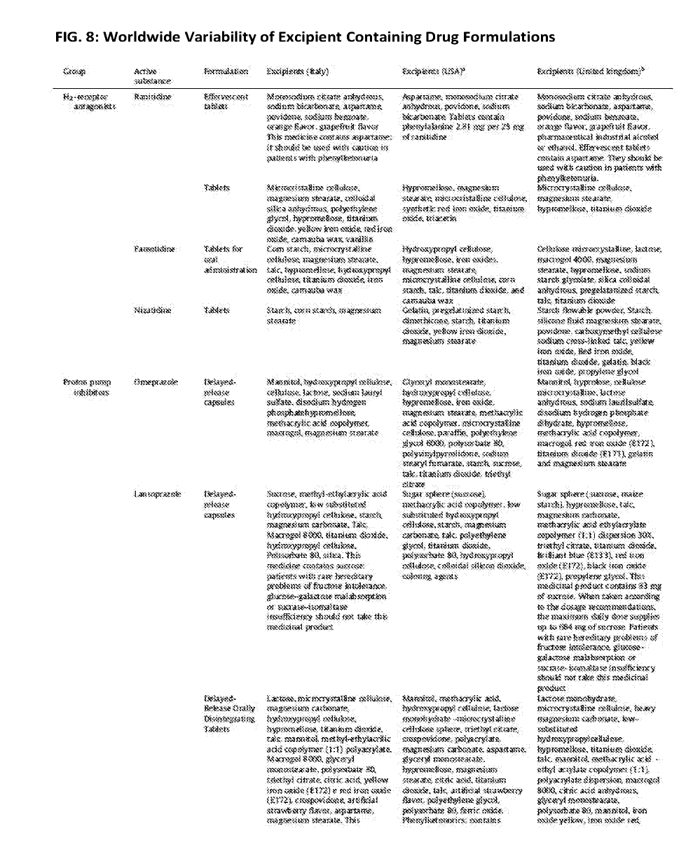

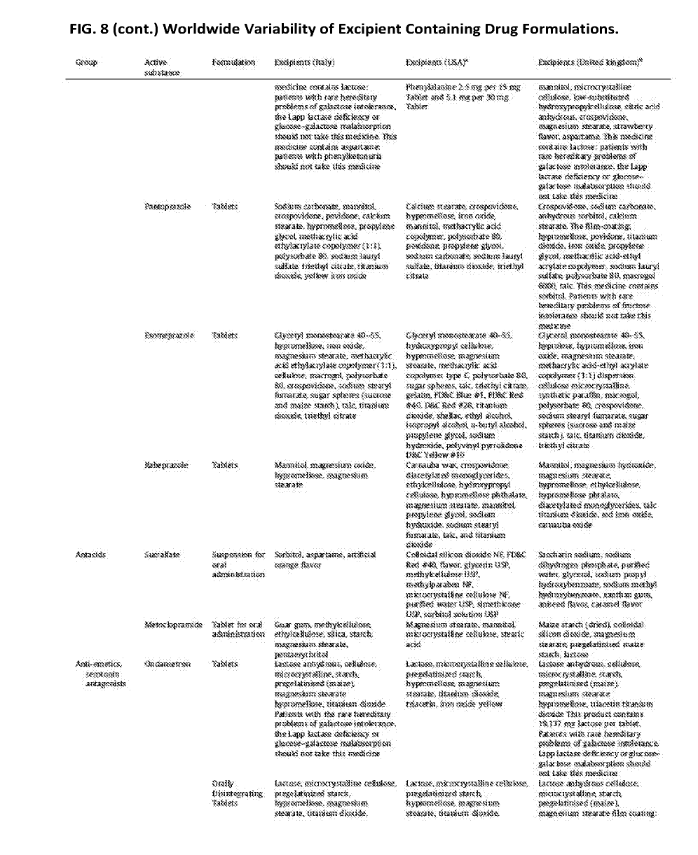

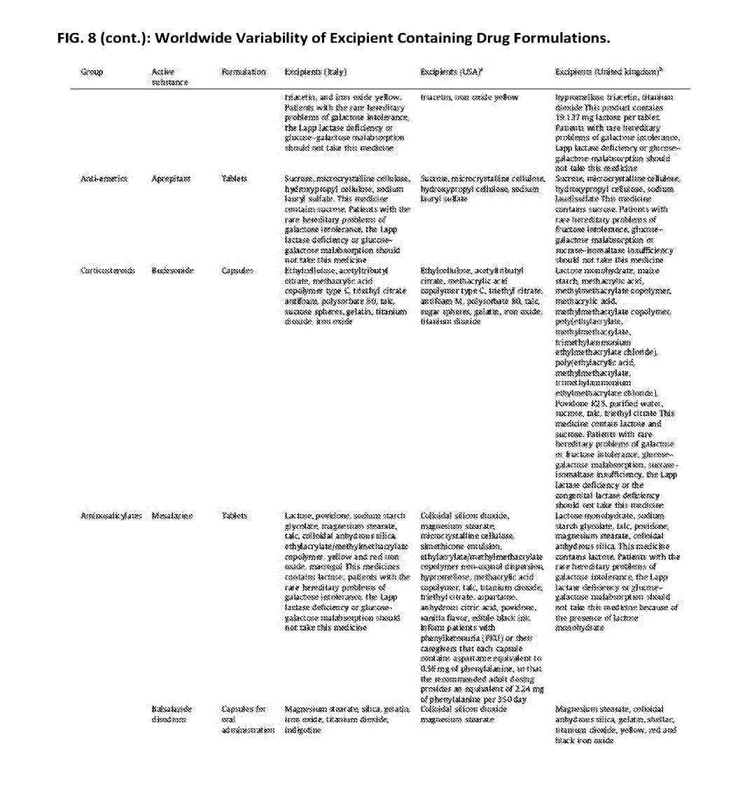

While regulatory efforts are legally constrained to characterizing the safety of individual drug excipients (e.g., lactose) and/or excipient contaminants (e.g., fururaldehyde contaminated lactose) with individual APIs (e.g. haloperidol), very little is known about the problem of excipient-excipient incompatibilities. This problem is compounded by the globalization of drug manufacturing in regions where regulatory oversight of GMP is either nonexistent or fraudulent FIG. 8. By way of example, known excipient contaminants can include: formaldehyde, reducing sugars, lactose phosphate, peroxides (e.g., hydrogen peroxide and hydroperoxide), free radicals, aldehydes, organic acids (e.g., formic acid, acetic acid, monochloro acetic acid) aldose, trace heavy metals, nitrites, and nitrates.

Highlighting this global problem, excipient impurities in drug products have become subject to greater scrutiny and various international and national guidelines, guidances and regulations have been proposed. Indeed, the reality of a global economy as a contributing factor to excipient stacking is highlighted by the fact that individual countries often have unique generic drug products with distinct excipient formulations.

Further stressing the importance of excipient toxicity as individual stand alone agents, federal drug regulations for topical drugs with inactive ingredients differing from the reference listed drug (RLD), or present in significantly different amounts, often specify that the applicant must identify and characterize the formulation differences and provide information demonstrating that the differences do not affect the safety or efficacy of the proposed drug product. One clear weakness of this regulatory approach is that topical drug excipients may “stack” with excipients from other co-administered common drug formulations (e.g., oral, intravenous, subcutaneous, etc.).

Drug compounding is another unrecognized contributory source of excipient stacking. When compounding extemporaneously, pharmacists must carefully choose the source for ingredients in order to assure the quality of the final preparation. United States Pharmacopeia (USP) Chapter <795> provides guidelines for selecting components used in compounding. The chapter states “a United States Pharmacopeia (USP), National Formulary (NF) or Food Chemicals Codex (FCC) substance is the recommended source of ingredients for compounding all preparations.” This includes both active ingredients and excipients.

USP <795> Pharmaceutical Compounding—Non-sterile Preparations also stresses that “compounders shall first attempt to use components manufactured in an FDA-registered facility.” When these components are not available, compounders should establish quality and purity by using information such as reputation and the Certificate of Analysis (CofA) provided by the manufacturer. USP <795> warns “when compounding with manufactured drug products the compounder shall consider all ingredients, including excipients, present in the drug product relative to the intended use of the compounded preparation and the effect of manipulating the drug product on the therapeutic appropriateness and stability of the components.”

One example of compounding where excipients can be quite problematic is preparing liquid suspensions from pre-manufactured tablets. Such compounded suspensions are often problematic because the excipients can increase viscosity and affect the rate and extent of settling, the dispersibility or homogeneity, and the pourability, as well as the palatability of the suspension. While many tablet and capsule drug products have generic equivalents available from multiple manufacturers, a major safety concern for compounding pharmacies is that the excipients in each manufacturer's product are not required to be identical. Pharmacies often purchase tablets and capsules from many different generic manufacturers depending on availability and pricing. Thus, the variability of compounding drugs may be best explained less by variability in the API and more by variability in the excipients.

Patient Populations at Risk of Excipient Stacking

By way of non-limiting examples, the pediatric population is particularly prone to excipient stacking. A contributing factor is a lack of comprehensive clinical toxicology studies in pediatric patients not only for the active ingredient, but also excipients. Some of the more common excipient-induced adverse events in children include: benzalkonium chloride-induced bronchospasm from antiasthmatic drugs, aspartame induced headache and seizures, saccharin-induced cross-sensitivity reactions in children with sulfonamide allergy, benzyl alcohol toxicity in neonates receiving high-dose continuous infusion with preserved medications, dye-related cross-reactions in children with aspirin intolerance, lactose-induced diarrhea, and propylene glycol-induced hyperosmolality and lactic acidosis.

While individual excipient safety data is publicly available in peer-reviewed scientific journals and government reports and databases, there is very limited pediatric excipient data that can be used for development of pediatric medicines. The Safety & Toxicity of Excipient For Pediatrics (i.e., STEP) database was created in response to the observation that there is no repository of excipients information (e.g., dose information, PK, etc.) that could provide a basis for screening and selecting excipients for use in pediatric product development and expedite further product-specific safety and toxicity studies. Problematic excipients listed in the current version of the STEP database capable of excipient stacking include: Alcohol, alpha-Cyclodextrin, Aspartame, Benzalkonium Chloride, Benzoic Acid, Benzyl Alcohol, beta-Cyclodextrin, Butylated Hydroxyanisole, Butylparaben, Carboxymethylcellulose, Carboxymethylcellulose Sodium, Crospovidone, Ethylparaben, gamma-Cyclodextrin, Glyceryl Monocaprylate [Capmul MCM C8], Hypromellose, Lactose, Mannitol, Methylparaben, Microcrystalline Cellulose, Polyethylene Glycol, Polysorbate 20, Polysorbate 40, Polysorbate 60, Polysorbate 65, Polysorbate 80, Povidone, Propylene Glycol, Propylparaben, Saccharin, Saccharin Sodium, Sodium Benzoate, Sodium Lauryl Sulfate, Sodium Metabisulphite, Sorbic acid, Sorbitol, Sucralose, Vitamin E Polyethylene Glycol Succinate, Xanthan Gum, Xylitol, Boric acid, Citric acid, Colloidal Silicon Dioxide, Butylated hydroxytoluene (BHT), L-proline, Polaxmers, Maltitol, Magnesium Stearate, Sucrose and Trehalose.

By further way of example, psychiatric and/or elderly patients may be highly susceptible to the adverse effects of excipient stacking because of emerging physiological connections between the gut-brain axis and neuropsychological disorders. Recently, a striking discovery revealed that gut bacteria consume the neurotransmitter GABA. Abnormally low levels of GABA are linked to depression and mood disorders. The authors speculate that this could help explain why the gut microbiome seems to affect mood.

Evidence additionally shows that schizophrenic patients may be predisposed to gut dysbiosis by demonstrating pre-existing increased gut bacterial translocation not attributed to medication. A better understanding of the interactions between the host immune system and gut bacteria should also shed light on how drug excipients may contribute to diseases by affecting the gut microbiome. The integrity of the gut mucosa is of critical importance to health. For example, excipient stacking may be particularly problematic in sensitive patient populations with underlying gut dysbiosis whereby the stacked excipient(s) function as a noxious trigger to exacerbate disease. Understanding which excipients influence the gut barrier, and which gut diseases are most susceptible to excipient stacking, may be of significant clinical value. Excipient stacking may also occur via ingestion of therapeutic bacterial strains containing excipients.

The elderly patient population is at an elevated risk of excipient stacking largely due to polypharmacy. Elderly persons often receive multiple drugs for many diseases (i.e., polypharmacy)—both from multiple prescribing physicians as well as other elderly individuals who are also taking medications (e.g., “drug sharing”). The most common shared prescription medications include opiates, central nervous system depressants and stimulants due to their addictive qualities.

Commonly shared opiates include:

Morphine (Kadian®, Avinza®)

Codeine (Tylenol® #2, 3, 4)

Oxycodone (OxyContin®, Percodan®, Percocet®)

Hydrocodone (Lortab®, Lorcet®, Vicodin®)

Propoxyphene (Darvon®)

Fentanyl (Duragesic®)

Hydromorphone (Dilaudid®)

Commonly shared CNS depressants include:

Barbiturates

Mephobarbital (Mebaral®)

Pentobarbital sodium (Nembutal®)

Butalbital (Fioricet®)

Benzodiazapines

Diazepam (Valium®)

Chlordiazepoxide HCl (Librium®)

Alprazolam (Xanax®)

Triazolam (Halcion®)

Estazolam (ProSom®)

Commonly shared stimulants include:

Amphetamine (Adderall®)

Dextroamphetamine (Adderall XR®, Dexedrine®)

Methylphenidate (Ritalin® and Concerta®)

While it is generally assumed that adverse drug interactions in the elderly are due to active ingredients, it is quite likely that adverse reactions are due, at least in part, to excipients. The high rates of comorbid illnesses in older populations, age-related changes in drug metabolism, and the potential for drug interactions may make any of these practices more dangerous than in younger populations. Further, a large percentage of older adults also use OTC medicines and dietary supplements, which (in addition to alcohol) could compound any adverse health consequences resulting from prescription drug abuse. One additional problem of the elderly is the unpredictability of their response to drugs. Self-medication with both prescribed and OTC drugs worsen this problem. Drug and excipient actions may be altered by impairment of the liver, kidneys and brain, or acute illness, or both. Adverse drug reactions often produce rapid and unduly severe illness in the old. Some antibacterial antibiotics like the sulphonamides are best avoided.

By way of examples, gut dysbiosis can include or be associated with: inflammatory bowel disease, irritable bowel syndrome (IBS), and coeliac disease, while extra-intestinal disorders include allergy, asthma, metabolic syndrome, cardiovascular disease, and obesity. Medications and conditions associated with gut dysbiosis include, but are not limited to: acid suppressor drugs (PPIs/antacids), alcohol, antibiotics, artificial sweeteners, BCP/hormones, birth control pills, bowel obstruction, chemotherapy, constipation, Crohn's disease, decreased motility, diabetes, diverticulosis, fatty foods, fistulas, gastric bypass surgery, hormone therapy, hypochlorhydria (low acid), hypothyroidism, immune deficiency, infections, low fiber, NSAIDs, pancreatic enzyme deficiency, parasites, post-operative changes, scleroderma, steroids, stress and sugar over-consumption.

By further way of example, patients with pre-existing gut sensitivities (genetic or otherwise) may be more susceptible to excipient stacking.

By further way of example, patients suffering from an autoimmune disorder(s) may be highly susceptible to the adverse effects of excipient stacking. This may be because of emerging effects of the gut-brain axis and neuropsychological disorders.

By further way of example, excipient stacking may occur in the elderly patient population who are taking multiple medications.

By further way of example, excipient stacking occurs in combinations of drug excipients and/or food/drink excipients and/or cosmetics.

By further way of example, excipient stacking may occur in a person genetically predisposed to excipient toxicity. For example, the azo dye tartrazine is known to be potentially dangerous in aspirin-intolerant individuals. Moreover, approximately 2% to 20% of asthmatics are sensitive to aspirin.

By way of example, excipient stacking may occur in a person with preexisting gut dysbiosis or susceptibility to gut dysbiosis due to antibiotic use, surgery, radiotherapy and other interventions generally known to affect the microbiome. This may occur by either the excipients in the antibiotic drug formulation itself and/or by antibiotic induced changes in the gut microbiota/intestinal flora.

While disclosed embodiments generally relate to drug formulations having reduced excipient content, the general principle of excipient stacking, and methods for reducing excipient stacking, can be applied to other drug formulations such as suspension, solid, injectable, topical, dermal, intranasal, intraoral, ophthalmic, rectal, or vaginal, pulmonary formulations or other formulations generally known to a person skilled in the art.

Non-limiting examples of excipients individually suspected of causing adverse events include: acacia, acesulfame, acesulfame potassium, acetic acid, acetone, acetyltributyl citrate, alcohol, alginic acid, alpha-tocopherol, aluminum chloride, aluminum chlorohydrex propylene glycol, aluminum hydroxide, aluminum lake dyes, aluminum oxide, aluminum silicate, aluminum stearate, aluminum sulfate, amide resin, aminobenzoate sodium, ammonia ammonio methacrylate copolymer, ammonio methacrylate copolymer type A, ammonio methacrylate copolymer type B, ammonio methacrylate copolymers, ammonium chloride, ammonium hydroxide, ammonium laureth-5 sulfate, ammonium phosphate dibasic, artificial flavor, artificial grape flavor, artificial mint flavor, ascorbic acid, ascorbyl palmitate, aspartame, aspartame powder, banana barium sulfate, benzalkonium chloride, benzoic acid, benzyl alcohol, betadex black currant, black currant flavor, black ink black pigment, blackberry, blue dye, butyl alcohol, butylated hydroxyanisole, butylated hydroxytoluene butylparaben, calcium, calcium carbonate, calcium phosphate, calcium phosphate dibasic anhydrous, calcium phosphate dihydrate dibasic, calcium silicate, calcium stearate, calcium sulfate, calcium sulfate anhydrous, calcium sulfate dehydrate, candelilla wax, candelilla wax powder, carbomer, carbomer 934, carbomer 934p, carbomer homopolymer type A, carbomer homopolymer type B, carbomer homopolymer type C, carboxymethylcellulose, carboxymethylcellulose calcium, carboxymethylcellulose sodium, carmine, carnauba wax, carrageenan, castor oil, castor wax, cellacefate, cellulose, cellulose acetate, cellulose compounds, cellulose powdered, cellulosic polymers, cetostearyl alcohol, cetyl alcohol, cetylpyridinium chloride, cherry, citric acid, citric acid anhydrous, citric acid monohydrate, cochineal, coconut oil colophony colorants, coloring agent, compressible sucrose, compressible sugar, confectioners sugar, copovidone, corn, corn oil, corn starch, corn syrup, corn syrup solids, corn-derived proteins, cottonseed oil, cranberry, croscarmellose sodium, croscarmellose sodium type A, crospovidone, cysteine hydrochloride, D&C Blue No. 1, D&C Green No. 5, D&C Red No. 21, D&C Red No. 22, D&C Red No. 27, D&C Red No. 27 Aluminum Lake, D&C Red No. 27 Lake, D&C Red No. 28, D&C Red No. 28 Aluminum Lake, D&C Red No. 30, D&C Red No. 30 Aluminum Lake, D&C Red No. 33, D&C Red No. 40, D&C Red No. 6, D&C Red No. 6 Lake, D&C Red No. 7, D&C Red No. 7 Calcium Lake, D&C Yellow No. 10, D&C Yellow No. 10 Aluminium Lake, D&C Yellow No. 10 Lake, D&C Yellow No. 5, D&C Yellow No. 6, dehydrated alcohol, dextrates, dextrose, dextrose monohydrate, dibasic calcium phosphate, dibutyl phthalate, dibutyl sebacate, dicalcium phosphate, diethyl phthalate, dihydroxyaluminum sodium carbonate, dimethicone, dimethylaminoethyl methacrylate-butyl methacrylate-methyl methacrylate copolymer, dimethylpolysiloxane docusate sodium, dyes, edetate calcium disodium, edetate disodium edible black ink, egg lecithin, erythrosine, erythrosine sodium, ethanolamine, ethyl acrylate-methyl methacrylate copolymer, ethyl alcohol, ethyl butyrate, ethyl isovalerate, ethylcellulose ethylcellulose (10 mPa·s), ethylcellulose (100 mPa·s), ethylcellulose (20 mPa·s), ethylcellulose (7 mPa·s), ethylcelluloses, ethylene glycol monoethyl ether, ethylvanillin, eudragit FD&C Blue No. 1, FD&C Blue No. 1 Aluminium Lake, FD&C Blue No. 1 Lake, FD&C Blue No. 2, FD&C Blue No. 2 Aluminium Lake, FD&C Blue No. 2 Lake, FD&C Green No. 3, FD&C Green No. 3 Aluminum Lake, FD&C Red No. 3, FD&C Red No. 4, FD&C Red No. 40, FD&C Red No. 40 Aluminium Lake, FD&C Red No. 40 Lake, FD&C Yellow No. 10, FD&C Yellow No. 10 Aluminum Lake, FD&C Yellow No. 10 Lake, FD&C Yellow No. 5, FD&C Yellow No. 5 Aluminum Lake, FD&C Yellow No. 5 Lake, FD&C Yellow No. 6, FD&C Yellow No. 6 Aluminum Lake, FD&C Yellow No. 6 Lake, ferric oxide, ferric oxide black, ferric oxide brown, ferric oxide orange, ferric oxide red, ferric oxide yellow, ferric oxides, ferrosoferric oxide, ferrous fumarate, ferrous oxide, flavor, flavors, fragrances, fumaric acid, gelatin, glucosamine, glucosamine hydrochloride, glutamic acid hydrochloride, glycerin, glycerol, glycerol monooleate, glycerol monostearate, glyceryl behenate, glyceryl distearate, glyceryl monooleate, glyceryl monostearate, glyceryl triacetate, glycine, glycolate, glycyrrhizin ammoniated, guar gum, hard gelatin capsule, hard paraffin, hydrochloric acid, hydrocloric acid, hydrogen peroxide, hydrogenated castor oil, hydrogenated cottonseed oil, hydrogenated soy oil, hydrogenated soybean oil, hydrogenated vegetable oil, hydroxyethyl cellulose, hydroxypropyl cellulose, hydroxypropyl methylcellulose, hydroxypropyl methylcellulose phthalate, hypromellose, hypromellose 2208, hypromellose 2208 (100 mPa·s), hypromellose 2208 (100000 mPa·s), hypromellose 2208 (15000 mPa·s), hypromellose 2208 (3 mPa·s), hypromellose 2208 (4000 mPa·s), hypromellose 2910, hypromellose 2910 (15 mPa·s), hypromellose 2910 (15000 mPa·s), hypromellose 2910 (3 mPa·s), hypromellose 2910 (5 mPa·s), hypromellose 2910 (50 mPa·s), hypromellose 2910 (6 mPa·s), hypromellose 2910 3cp, hypromellose 2910 50cp, hypromellose 2910 5cp, hypromellose 2910 6cp, hypromellose 3cp, hypromellose 5cp, hypromellose 6cp, hypromellose phthalate, hypromelloses, indigotindisulfonate sodium, iron, isobutylparaben, isopropyl, isopropyl alcohol, lactitol, lactitol monohydrate, lactose, lactose anhydrous, lactose hydrous, lactose monohydrate, lecithin, lemon oil, leucine, light mineral oil, low substituted hydroxypropyl cellulose, magnesium, magnesium aluminum silicate, magnesium carbonate, magnesium hydroxide, magnesium oxide, magnesium oxide heavy, magnesium silicate, magnesium stearate, magnesium trisilicate, maleic acid, malic acid, maltodextrin, mannitol, medium-chain triglycerides, meglumine, menthol, methacrylic acid, methacrylic acid-ethyl acrylate copolymer (1:1) type a, methacrylic acid-methyl methacrylate copolymer (1:1), methacrylic acid-methyl methacrylate copolymer (1:2), methacrylic acid copolymer, methacrylic acid copolymer type B, methanol, methyl alcohol, methyl cinnamate, methyl methacrylate, methylcellulose, methylcellulose (100 mPa·s), methylcellulose (15 mPa·s), methylcellulose (400 mPa·s), methylene chloride, methylparaben, methylparaben sodium, microcrystalline cellulose, microcrystalline wax, mineral oil, mint, mint cream flavor, mint menthol, modified corn starch, monosodium citrate, natural and artificial orange flavor, natural flavor, natural mint flavor, natural peppermint flavor, natural resin, nonoxynol-100, oleic acid, olive oil, opacode black, orange cream flavor, orange juice, orange oil, orange-pineapple flavor, other ingredients known to those skilled in the art, palm kernel oil, paraffin, partially hydrogenated soybean and palm oils, peanut oil, peppermint, peppermint flavor, peppermint oil, pharmaceutical glaze, phenylalanine, phosphoric acid, piperazine, polacrilin potassium, polacrilin sodium, poloxamer, poloxamer 188, poloxamer 407, polyacrylate dispersion 30%, polycarbophil, polydextrose, polyethylene glycol, polyethylene glycol 1450, polyethylene glycol 300, polyethylene glycol 3000, polyethylene glycol 3350, polyethylene glycol 400, polyethylene glycol 4000, polyethylene glycol 600 polyethylene glycol 6000, polyethylene glycol 800, polyethylene glycol 8000, polygalacturonic acid, polyplasdone×1, polysorbate, polysorbate 20, polysorbate 80, polyvinyl acetate, polyvinyl alcohol, polyvinylpyrrolidone, potassium, potassium bicarbonate, potassium bitartrate, potassium carbonate, potassium carbonate anhydrous, potassium chloride, potassium gluconate, potassium hydroxide, potassium sorbate, potato starch, povidone, povidone k12, povidone k25, povidone k29/32, povidone k30, povidone k90, precipitated calcium carbonate, pregelatinized corn starch, pregelatinized starch, propyl gallate, propylene glycol, propylene glycol alginate, propylparaben, propylparaben sodium, raspberry, raw sugar, riboflavin, rice starch, saccharin, saccharin sodium, sd-45 alcohol, sda-3a alcohol, sesame oil, shellac, silicified microcrystalline cellulose, silicon dioxide, silicon dioxide colloidal, silicone, simethicone, simethicone emulsion, sodium, sodium alginate, sodium ascorbate, sodium benzoate, sodium bicarbonate, sodium carbonate, sodium carbonate monohydrate, sodium caseinate, sodium chloride, sodium citrate, sodium citrate dehydrate, sodium glycolate, sodium hydroxide, sodium laureth sulfate, sodium lauryl sulfate, sodium lauryl sulphate, sodium metabisulfite, sodium monolaurate, sodium phosphate, sodium phosphate dibasic, sodium propionate, sodium starch glycolate, sodium starch glycolate type A potato, sodium stearate, sodium stearyl fumarate, sodium thioglycolate, sodium tripolyphosphate, sorbic acid, sorbitan, sorbitan monolaurate, sorbitan monooleate, sorbitol, sorbitol special, soya lecithin, soybean oil, spearmint, starch, stearic acid, stearyl alcohol, strawberry, strawberry guarana flavor, strong ammonia solution, succinic acid, sucralose, sucrose, sucrose stearate, sugar 6× powder, sugar spheres, sunflower oil, synthetic ferric oxide, synthetic ferric oxide black, synthetic ferric oxide red, synthetic ferric oxide yellow, synthetic ferric oxides, tapioca starch, tartaric acid, tartrazine, taurine, TIMERx-N, titanium dioxide, titanium oxide, tragacanth, triacetin, tribehenin, tricalcium phosphate, triethyl citrate, trimyristin, trisodium citrate anhydrous, trisodium citrate dehydrate, tromethamine, tropical blend flavor, vanilla, vanilla flavor, vanillin, vitamin e, water, wax, wheat starch, white wax, xanthan gum, xylitol, yellow wax, zinc gluconate, and zinc stearate.

It should be emphasized that the foregoing description and examples have been presented for purpose of providing a clear understanding of the invention. The description is not intended to be exhaustive or to limit the invention to the precise examples disclosed.

Obvious modifications or variations by one with skill in the art are possible in light of the above teachings without departing from the spirit and principles of the invention. All such modifications and variations are intended to be within the scope of the present invention.

### LITERATURE

1. http://www.gastroorg/patient-center/diet-medications/food-allergies-fructose-intolerance-and-lactose-intolerance#Lactose%20Intolerance
2. http://digestive.niddk.nih.gov/ddiseases/pubs/lactoseintolerance/3.
3. http://www.stevecarper.com/li/dairy_facts.htm
4. Matthews S B, Waud J P, Roberts A G, Campbell A K. Systemic lactose intolerance: a new perspective on an old problem: Postgrad Med J 2005; 81:167-173 (click to download pdf).
5. Montalto M, Curigliano V, Santoro L, Vastola M, Cammarota G, Manna R, Gasbarrini A, Gasbarrini G. Management and treatment of lactose malabsorption. World J Gastroenterol 2006; 12:187-191 (click to download pdf). Mattar R, de Campos Mazo D F, Carrilho F J. Lactose intolerance: diagnosis, genetic, and clinical factors. Clin Exp Gastroenterol. 2012; 5:113-121.
6. Heyman M B. Lactose intolerance in infants, children and adolescents. Pediatrics 2006; 118:1279-1286 (click to download pdf).
7. Bartra J et al. From Pollinosis to Digestive Allergy. J Investig Allergol Clin Immunol 2009; 19(Suppl 1):3-10 (click to download pdf).
8. Ferreira F, Hawranek T, Gruber P, Wopfner N, Mari A. Allergic cross-reactivity: from gene to clinic. Allergy 2004; 59:243-267 (click to downloadpdf).
9. Konstantinou G N, Grattan C E H. Food contact hypersensitivity syndromes: the mucosal contact urticaria paradigm. Clin Exp Dermatol 2008; 33:383-389 (click to download pdf).
10. MaintzL, Novak N. Histamine and histamine intolerance. Am J Clin Nutr 2007; 85:1185-1196 (click to download pdf).
11. Schwelberger H G. Histamine intolerance: a metabolic disease? Inflamm Res 2010:59(suppl 2):219-221 (click to download pdf).
12. Hypersensitivity Reaction After Inhalation of a Lactose-Containing Dry Powder Inhaler J Pediatr Pharmacol Ther 2014; 19(3):206-211 Janie Robles, PharmD1 and Lesley Motheral, MD2
13. Bhattacharyya, Lokesh; Schuber, Stefan; Sheehan, Catherine; William, Roger (2006). “Excipients: Background/Introduction”. In Katdare, Ashok; Chaubal, Mahesh. Excipient Development for Pharmaceutical, Biotechnology, and Drug Delivery Systems.
14. (21 CFR § 74.1705) “This product contains FD&C Yellow No. 5 (tartrazine) which may cause allergic-type reactions (including bronchial asthma) in certain susceptible persons. Although the overall incidence of tartrazine sensitivity in the general population is low, it is frequently seen in patients who also have aspirin hypersensitivity.”
15. Guidance for Industry—Nonclinical Studies for the Safety Evaluation of Pharmaceutical Excipients. U.S. Department of Health and Human Services, Food and Drug Administration, Center for Drug Evaluation and Research (CDER), Center for Biologics Evaluation and Research (CBER), May 2005 J:\!GUIDANC\5544fnIcln2.doc Apr. 15, 2005
16. See The Safety and Utility of Skin Testing With XOLAIR® (Omalizumab) and Placebo Omalizumab (Formulation Excipients) ClinicalTrials.gov Identifier: NCT00777764
17. Phase 2, Randomized, Double Blind, Placebo Controlled Multicenter Study of Autologous MSC-NTF Cells in Patients With ALS (NurOwn) ClinicalTrials.gov Identifier: NCT02017912
18. Effect of the Consumption of a Probiotic Strain on the Prevention of Post-operative Recurrence in Crohn's Disease ClinicalTrials.gov Identifier: NCT01698970
19. Toxicity of Excipients—A Food and Drug Administration Perspective, Robert E. Osterberg and
20. Norman A. International Journal of Toxicology, September/October 2003; vol. 22, 5: pp. 377-380.
21. Trends in Excipient Safety Evaluation, Robert E. Osterberg, Christopher C. DeMerlis, David W. Hobson, and Timothy J. McGovern International Journal of Toxicology, December 2011; vol. 30, 6: pp. 600-610.
22. Guidance for Industry Limiting the Use of Certain Phthalates as Excipients in CDER-Regulated Products, U.S. Department of Health and Human Services Food and Drug Administration
23. Center for Drug Evaluation and Research (CDER) December 2012 CMC
24. Guidance for Industry Nonclinical Studies for the Safety Evaluation of Pharmaceutical Excipients, U.S. Department of Health and Human Services, Food and Drug Administration
25. Center for Drug Evaluation and Research (CDER) Center for Biologics Evaluation and Research (CBER) May 2005 Pharmacology/Toxicology
26. Gonzalo Garijo M A, Duran Quintana J A, Bobadilla Gonzalez P, Maiquez Asuero P, Anaphylactic shock following povidone, The Annals of Pharmacotherapy, 1996, 30(1), 37-40
27. Assal C, Watson P Y, Angioedema as a hypersensitivity reaction to polyethylene glycol oral electrolyte solution, Gastrointestinal Endoscopy, 2006, 64(2), 294-295
28. Van Gossum A, Adler M, Complications of bowel cleansing with polyethylene glycol electrolyte solution, Gastrointestinal Endoscopy, 1999, 49(6), 818-818
29. Barrett J S, Irving P M, Shepherd S J, Muir J G, Gibson P R, Comparison of the prevalence of fructose and lactose malabsorption across chronic intestinal disorders, Alimentary Pharmacology and Therapeutics, 2009, 30(2), 165-174
30. Michel O, Naeije N, Bracamonte M, Duchateau J, Sergysels R (May 1984). “Decreased sensitivity to tartrazine after aspirin desensitization in an asthmatic patient intolerant to both aspirin and tartrazine”. Annals of Allergy 52 (5): 368-70.
31. AMERICAN ACADEMY OF PEDIATRICS, Committee on Drugs “Inactive” Ingredients in Pharmaceutical Products: Update PEDIATRICS Vol. 99 No. 2 Feb. 1997
32. The safety of pharmaceutical excipients. Giorgio Pifferi, Patrizia Restani. Institute of Pharmaceutical and Toxicological Chemistry, V.le Abruzzi 42, Milan, Italy. Department of Pharmacological Sciences, Via Balzaretti 9, Milan, Italy
33. Pharmaceutical Excipients: A review Shilpa P Chaudhariand Pradeep S Patil Marathwada Mitra Mandal's College of Pharmacy, Thergaon, Pune, Maharashtra, India. IJAPBC—Vol. 1(1), January-March, 2012 INTERNATIONAL JOURNAL OF ADVANCES IN PHARMACY, BIOLOGY AND CHEMISTRY.
34. Regul Toxicol Pharmacol. 2011 June; 60(1):93-105. doi: 10.1016/j.yrtph.2011.02.010. Epub 2011 Feb. 24. Excipients in medicinal products used in gastroenterology as a possible cause of side effects. Ursino MG1, Poluzzi E, Caramella C, De Ponti F.
35. Golightly, L. K., Smolinske, S. S., Bennett, M. L., Sutherland III, E. W., Rumack, B. H., 1988. Pharmaceutical excipients. Adverse effects associated with inactive ingredients in drug products (Part I). Med. Toxicol. Adverse Drug Exp. 3 (2), 128-165.
36. EMA, 2003. Excipients in the label and package leaflet of medicinal products for human use (EUDRALEX 3BC7A).
37. EMA, 2005. EMA/CHMP/PEG/194810/2005. Reflection paper: formulations of choice for the paediatric population.
38. EMA, 2009. A guideline on summary of product characteristics, September 2009, October 2005.
39. FDA, 2005. Guidance for Industry Nonclinical studies for Safety evaluation of Pharmaceutical Excipients, May 2005.
40. FDA, 2010. Inactive ingredients database. Available online: <http://www.fda.gov/Drugs/InformationOnDrugs/ucm113978.htm> (accessed on 31.12.10).
41. Recent Patents on Drug Delivery & Formulation 2013, 7, 73-83 73, Co-Processed Excipients: A Patent Review. Nidhi Garg, Harish Dureja and Deepak Kaushik.

1. Excipient reduced pharmaceutical drug formulations obtained by: (i) comparing excipient profiles from two or more co-administered solid drug formulations, (ii) identifying the excipient or excipients common to said two or more co-administered solid drug formulations, (iii) reformulating one or more of said co-administered solid drug formulations without the excipient or excipients common to said two or more co-administered drug formulations.

2. The excipient reduced pharmaceutical drug formulation of claim 1, wherein the formulation is capable of being administered orally, wherein the formulation is selected from a liquid, a suspension, and an emulsion.

3. (canceled)

4. (canceled)

5. (canceled)

6. The excipient reduced pharmaceutical drug formulation of claim 1, wherein the formulation is capable of being administered to the eye, wherein the formulation is selected from a liquid, a suspension, and an emulsion.

7. (canceled)

8. (canceled)

9. (canceled)

10. The excipient reduced pharmaceutical drug formulation of claim 1, wherein the formulation is capable of being administered to the nose, wherein the formulation is selected from a liquid, a suspension, and an emulsion.

11. (canceled)

12. (canceled)

13. (canceled)

14. The excipient reduced pharmaceutical drug formulation of claim 1, wherein the formulation is capable of being administered to the ear, wherein the formulation is selected from a liquid, a suspension, and an emulsion.

15. (canceled)

16. (canceled)

17. (canceled)

18. The drug formulation of claim 1, wherein the formulation is capable of being administered to the rectum, wherein the formulation is selected from a liquid, a suspension, and an emulsion.

19. (canceled)

20. (canceled)

21. (canceled)

22. The excipient reduced pharmaceutical drug formulation of claim 1, wherein the formulation is capable of being administered to the vagina, wherein the formulation is selected from a liquid, a suspension, and an emulsion.

23. (canceled)

24. (canceled)

25. (canceled)

26. The excipient reduced pharmaceutical drug formulation of claim 1, wherein the formulation is capable of being administered parenterally, wherein the formulation is selected from a liquid, a suspension, and an emulsion.

27. (canceled)

28. (canceled)

29. (canceled)

30. The excipient reduced pharmaceutical drug formulation of claim 1, wherein the formulation is capable of being administered intravenously.

31. A method of identifying an excipient or excipients contributing to adverse events in an individual comprising comparing adverse events associated with co-administration of two or more drug pharmaceutical formulations having at least one common excipient to the adverse events associated with co-administration of two or more drug pharmaceutical formulations lacking said common excipient.

32. The method of claim 31 wherein said two or more drug pharmaceutical formulations contain the same active ingredient or ingredients.

33. The method of claim 31 wherein said two or more drug pharmaceutical formulations contain a different active ingredient or ingredients.

34. The method of claim 33 wherein said two or more drug pharmaceutical formulations containing a different active ingredient or ingredients have no known chemical cross-reactivity.

35. A method of identifying an excipient or excipients contributing to adverse events in an individual comprising comparing adverse events associated with repeated daily administration of a drug pharmaceutical formulation containing an excipient or excipients to the adverse events associated with repeated daily administration of a drug pharmaceutical formulation having the same active ingredient but lacking said excipient or excipients.

36. The method of claim 35 wherein said drug pharmaceutical formulation is an OTC drug formulation.

37. A method of avoiding excipient stacking in an individual comprising administrating to said individual one or more pharmaceutical formulations lacking an excipient or excipients previously identified as contributing to adverse events.

38. The method of claim 37 wherein said formulation is selected from the group consisting of a liquid formulation, a liquid elixir formulation, a liquid suspension formulation, a liquid emulsion formulation, an oral liquid formulation, an oral elixir formulation, an oral suspension formulation and an oral emulsion formulation.

39. (canceled)

40. (canceled)

41. (canceled)

42. (canceled)

43. (canceled)

44. (canceled)

45. (canceled)

46. A method of identifying an excipient or excipients contributing to adverse events in a non-human animal comprising comparing adverse events associated with co-administration of two or more drug pharmaceutical formulations having at least one common excipient to the adverse events associated with co-administration of two or more drug pharmaceutical formulations lacking said common excipient.

47. The method of claim 46 wherein said non-human mammal is selected from the group consisting of a canine, a feline, a bovine and an equine.

48. (canceled)

49. (canceled)

50. (canceled)
